(19) United States
(12) Patent Application Publication
(10) Pub. No.: US 2008/0306472 A1
(43) Pub. Date: Dec. 11, 2008

(54) LASER APPARATUS WITH INTEGRATED FOOTSWITCH CONTROL

(75) Inventors: BRIAN PRYOR, EWING, NJ (US); SEAN XIAOLU WANG, WILMINGTON, DE (US)

Correspondence Address: FRANK F. TIAN, 331-4A THIRD AVENUE, LONG BEACH, NJ, 07740, US

(73) Assignee: BWT PROPERTY, INC., Newark, DE (US)

(21) Appl. No.: 12/133,789

(22) Filed: Jun. 5, 2008

Related U.S. Application Data
(60) Provisional application No. 60/942,103, filed on Jun. 5, 2007.

(51) Int. Cl. A61B 18/20

(52) U.S. Cl. 606/10

(57) ABSTRACT

A laser apparatus is disclosed. The laser apparatus can be placed on the ground of an operation site to save precious desktop space. The laser apparatus comprises a footswitch module physically integrated or combined with the laser module for controlling the status of the laser module. The laser apparatus further comprises a miniature control unit, which can be clipped onto the pocket of the operator or be integrated or combined on the hand piece of the laser apparatus for controlling and displaying status of the laser module.

### REFERENCE TO RELATED APPLICATIONS

This application claims an invention which was disclosed in Provisional Application Number 60/942,103, filed Jun. 5, 2007 entitled “LASER APPARATUS WITH INTEGRATED FOOTSWITCH CONTROL”. The benefit under 35 USC §119(e) of the United States provisional application is hereby claimed, and the aforementioned application is hereby incorporated herein by reference.

### FIELD OF THE INVENTION

This invention generally relates to a laser apparatus, and more specifically to a medical laser apparatus with integrated or combined footswitch control.

### BACKGROUND

Medical lasers are widely used as surgical or therapeutic tools. Conventional medical lasers are typically controlled with foot operating switches such as foot pedals through wired connections. Some examples can be found in U.S. Pat. Nos. 5,166,513, 5,568,859, and 5,580,347. From a practical point of view, these conventional medical laser apparatus suffer from the following shortcomings. First, the laser apparatus are desktop devices that occupy a lot of precious surface area in an office space having a distance away from the foot switch. Second, it naturally follows that the electrical wire that is used to connect the medical laser with its foot pedal may cause a safety issue as the medical personnel may trip over the wire. In case where multiple medical devices are placed in the same office, the multiple control units and electrical wires may cause confusion and mishandling.

To solve these problems, it had been proposed to replace the wired connection with a remote wireless control, such as that disclosed by Larson in U.S. Pat. No. 6,066,129; and by Hamel in U.S. Pat. Appl. No. 20050251228. However, the wireless control may be less robust in its resistance to electromagnetic interference (EMI).

There thus exists a need for an improved medical laser apparatus which occupies less desired desktop space and provides convenient output control with enhanced safety features.

### SUMMARY OF THE INVENTION

According to one aspect of the present invention, a compact medical laser apparatus is provided which comprises an integrated or combined footswitch for output control by integrating or combining a foot operated switch with the laser module. This is made possible by the advancement of new and compact solid state laser modules. The footswitch is embedded into the medical laser apparatus so that no further wire connection between the foot switch and the laser module is required. The entire medical laser apparatus can be placed on the ground to save precious desktop space.

According to another aspect of the present invention, a miniature control unit is provided for the medical laser, which can be suitably clipped onto the pocket of the medical personnel or be integrated or combined on the hand piece of the medical laser for controlling and displaying related parameters of the medical laser, such as on/off status, power level, etc.

A laser apparatus that can be placed on the ground of an operation site is provided. The laser apparatus comprises a laser module for producing a laser beam; a footswitch module physically integrated or combined with the laser module for controlling a status of the laser module; and an optical waveguide for delivering the laser beam.

A method for making a laser apparatus that can be placed on the ground of an operation site is provided.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one preferred embodiment of the present invention, wherein a compact medical laser is integrated or combined together with a footswitch and a miniature control unit to control the on/off status and power level of the laser.

FIG. 2 illustrates a variation of the medical laser apparatus of FIG. 1.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a laser apparatus with integrated or combined footswitch control. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

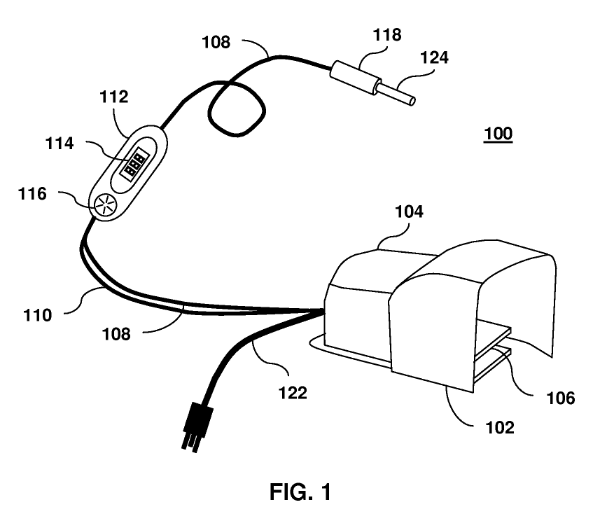

In the first preferred embodiment of the present invention as shown in FIG. 1, the compact medical laser apparatus 100 comprises a laser module 104 and a footswitch module 102 that are physically (e.g. mechanically and electrically) integrated or combined together. The laser module 104 and the footswitch module 102 share the same housing, which can be placed on the floor of an office to save precious desktop space. The electronic circuit of the footswitch (not shown) is directly connected, integrated, or combined with the driver and control circuit board (also not shown) of the laser module 104 so that no additional external wiring is required between the footswitch module 102 and the laser module 104. A wall plug 122 is used to supply electrical power to the combined laser module 104 and the footswitch module 102. As a slight variation, a rechargeable or replaceable battery may be embedded inside the laser apparatus as the power supply. The operator of the medical laser apparatus 100 may control on/off status, power level as well as other parameters of the laser module 104 through a foot pedal 106 of the footswitch module 102. The output laser beam of the laser module 104 is delivered through an optical fiber 108 (or other kinds of optical waveguides) with its proximal end connected to the output port of the laser module (not shown). The optical fiber 108 distal end is connected to a hand piece or wand 118, which outputs the laser beam to the targeted biological tissue (not shown) to be treated. The output optical waveguide 124 of the hand piece 118 can be designed as a detachable and disposable element for ease of disinfection or for controlling the number of treatments performed with each output optical waveguide 124.

The medical laser apparatus 100 further comprises a miniature control unit 112, which is used in conjunction with the footswitch module 102 to control the laser module 104. The control unit 112 comprises a display unit 114 for displaying the status of the laser module 104 and a knob (or a multi-position switch) 116 for controlling the laser's power level and on/off status. The control unit 112 communicates with the laser module 104 through an electrical wire 110 that is bundled together with the optical fiber 108. The setting of the knob 116, which is related to the power level of the laser, is converted into an electrical signal and sent to the laser module 104 through the electrical wire 110. This knob setting is used in conjunction with the setting of the foot pedal 106 to control the status of the laser module 104. For example, the laser module 104 can be turned on only when both the foot pedal 106 and the knob 116 are put at ‘on’ position. After the laser module 104 is turned on, the setting of the knob 116 is used to control the power level of the laser. The measured power level of the laser by a photo detector in the laser module (not shown) is transmitted through the electrical wire 110 to the control unit 112 and displayed on the display unit 114. In a slight variation of the present embodiment, the control unit 112 may be integrated or combined on the hand piece or wand 118 to save more space.

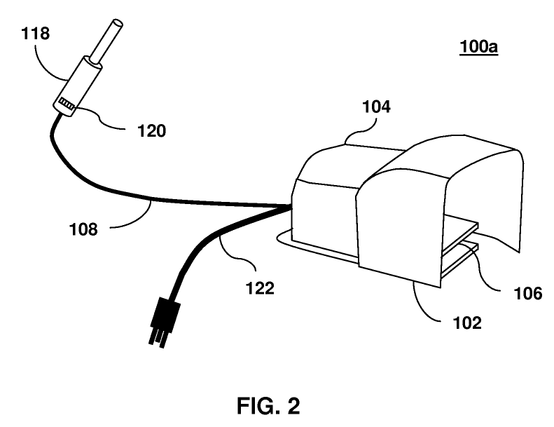

In a variation of the present embodiment 100a as shown in FIG. 2, the miniature control unit is replaced with an optical shutter and attenuator 120 that is integrated or combined on the hand piece or wand 118 of the laser apparatus 100. The optical shutter and attenuator 120 can control the on/off status and power level of the laser beam through opto-mechanical means. Thus no electrical wiring is required between the hand piece 118 and the laser module 104.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A laser apparatus that can be placed on the ground of an operation site, the laser apparatus comprising:
a laser module for producing a laser beam;
a footswitch module physically integrated or combined with the laser module for controlling a status of the laser module; and
an optical waveguide for delivering the laser beam.

2. The medical laser apparatus of claim 1, further comprising a control module for controlling the status of the laser module in conjunction with the footswitch module, wherein the control module is operable with the hand of an operator of the laser apparatus.

3. The medical laser apparatus of claim 2, wherein the control module utilizes electronic control.

4. The medical laser apparatus of claim 2, wherein the control module utilizes opto-mechanical control.

5. The medical laser apparatus of claim 2, wherein the control module further comprising a display unit for displaying the status of the laser module.

6. The medical laser apparatus of claim 1, wherein the laser module comprises compact solid state laser modules.

7. A method for making a laser apparatus that can be placed on the ground of an operation site, the laser apparatus comprising:
providing a laser module for producing a laser beam;
providing a footswitch module physically integrated or combined with the laser module for controlling a status of the laser module; and
providing an optical waveguide for delivering the laser beam.

8. The method of claim 7, further comprising providing a control module for controlling the status of the laser module in conjunction with the footswitch module, wherein the control module is operable with the hand of an operator of the laser apparatus.

9. The method of claim 8, wherein the control module utilizes electronic control.

10. The method of claim 8, wherein the control module utilizes opto-mechanical control.

11. The method of claim 8, wherein the control module further comprising a display unit for displaying the status of the laser module.

12. The method of claim 7, wherein the laser module comprises compact solid state laser modules.
